(19) United States
(12) Patent Application Publication
(10) Pub. No.: US 2006/0188481 A1
(43) Pub. Date: Aug. 24, 2006

(54) Methods for prophylactically or therapeutically treating an animal for an ocular-related disorder

(75) Inventor: Keisuke Mori, Maebashi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Saitama Medical School, Iruma-gun (JP)

(21) Appl. No.: 11/358,838

(22) Filed: Feb. 21, 2006

Related U.S. Application Data
(60) Provisional application No. 60/654,980, filed on Feb. 22, 2005.

(51) Int. Cl. A61K 48/00; C12N 15/861

(52) U.S. Cl. 424/93.2; 514/44; 435/456

(57) ABSTRACT

The invention is directed to a method of prophylactically or therapeutically treating an animal for an ocular-related disorder. The method comprises administering to an eye of the animal an expression vector comprising a nucleic acid sequence encoding an adenoviral-responsive gene product. The expression vector transduces a host cell and the nucleic acid sequence is expressed to produce the adenoviral-responsive gene product to treat prophylactically or therapeutically the ocular-related disorder. The invention also provides a method of reducing or inhibiting angiogenesis or photoreceptor cell loss in an eye. The method comprises administering to the eye an expression vector comprising a nucleic acid sequence encoding an adenoviral-responsive gene product. The expression vector transduces a host cell and the nucleic acid sequence is expressed to produce the adenoviral-responsive gene product thereby reducing or inhibiting angiogenesis or photoreceptor cell loss in the eye.

### CROSS-REFERENCE TO RELATED APPLICATIONS

This patent application claims the benefit of U.S. Provisional Patent Application No. 60/654,980, filed Feb. 22, 2005.

### FIELD OF THE INVENTION

The invention relates to a method of prophylactically or therapeutically treating an animal for an ocular-related disorder.

### BACKGROUND OF THE INVENTION

An overwhelming majority of the world's population will experience some degree of vision loss in their lifetime. Vision loss affects virtually all people regardless of age, race, economic or social status, or geographical location. Ocular-related disorders, while often not life threatening, necessitate life-style changes that jeopardize the independence of the afflicted individual. Vision impairment can result from most all ocular disorders, including diabetic retinopathies, proliferative retinopathies, retinal detachment, toxic retinopathies, retinal vascular diseases, retinal degenerations, vascular anomalies, age-related macular degeneration and other acquired disorders, infectious diseases, inflammatory diseases, ocular ischemia, pregnancy-related disorders, retinal tumors, choroidal tumors, choroidal disorders, vitreous disorders, trauma, cataract complications, dry eye, and inflammatory optic neuropathies.

Leading causes of severe vision loss and blindness are ocular-related disorders wherein the vasculature of the eye is damaged or insufficiently regulated. Ocular-related diseases comprising a neovascularization aspect are many and include, for example, exudative age-related macular degeneration, diabetic retinopathy, corneal neovascularization, choroidal neovascularization, neovascular glaucoma, cyclitis, Hippel-Lindau Disease, retinopathy of prematurity, pterygium, histoplasmosis, iris neovascularization, macular edema, glaucoma-associated neovascularization, and the like. Damage of the retina, i.e., retinal detachment, retinal tears, or retinal degeneration, is directly connected to vision loss. While a common cause of retinal detachment, retinal tears, and retinal degeneration is abnormal, i.e., uncontrolled, vascularization of various ocular tissues, this is not always the case. Atrophic complications associated with age-related macular degeneration, nonproliferative diabetic retinopathy, and inflammatory ocular damage are not associated with neovascularization, but can result in severe vision loss if not treated. Disorders associated with both neovascular and atrophic components, such as age-related macular degeneration and diabetic retinopathy, are particularly difficult to treat due to the emergence of a wide variety of complications.

For many ocular-related disorders, no efficient therapeutic options currently are available. Laser photocoagulation involves administering laser burns to various areas of the eye and is used in the treatment of many neovascularization-linked disorders. For example, focal macular photocoagulation is used to treat areas of vascular leakage outside the macula (Murphy, Amer. Family Physician, 51(4), 785-796 (1995)). Similarly, neovascularization, in particular, advanced proliferative retinopathy, is commonly treated with scatter or panretinal photocoagulation. Laser treatment does not guarantee that vision loss will be attenuated. In fact, many patients afflicted with age-related macular degeneration eventually experience severe vision loss in spite of treatment. Other treatment options for ocular-related disorders include thermotherapy, radiation therapy, surgery, e.g., macular translocation, removal of excess ocular tissue, drug therapy, and the like. However, in most cases, all available treatment options have limited therapeutic effect, require repeated, costly procedures, and/or are associated with dangerous side-effects.

Given the prevalence of ocular-related disorders, there remains a need for an effective prophylactic and therapeutic treatment of ocular-related disorders. Accordingly, the invention provides materials and methods for achieving a beneficial effect in the eye, such as inhibiting or reducing angiogenesis or preventing photoreceptor cell loss. This and other advantages of the invention will become apparent from the detailed description provided herein.

### BRIEF SUMMARY OF THE INVENTION

The invention is directed to a method of prophylactically or therapeutically treating an animal for an ocular-related disorder. The method comprises administering to an eye of the animal an expression vector comprising a nucleic acid sequence encoding an adenoviral-responsive gene product. The expression vector transduces a host cell and the nucleic acid sequence is expressed to produce the adenoviral-responsive gene product to treat prophylactically or therapeutically the ocular-related disorder. The invention further provides a method of reducing or inhibiting angiogenesis or photoreceptor cell loss in an eye. The method comprises administering to the eye an expression vector comprising a nucleic acid sequence encoding an adenoviral-responsive gene product. The expression vector transduces a host cell and the nucleic acid sequence is expressed to produce the adenoviral-responsive gene product thereby reducing or inhibiting angiogenesis or photoreceptor cell loss in the eye.

### DETAILED DESCRIPTION OF THE INVENTION

The invention is directed to a method of prophylactically or therapeutically treating an animal for an ocular-related disorder. The method comprises administering to an eye of the animal an expression vector comprising a nucleic acid sequence encoding an adenoviral-responsive gene product. The expression vector transduces a host cell and the nucleic acid sequence is expressed to produce the adenoviral-responsive gene product to treat prophylactically or therapeutically the ocular-related disorder. The invention is predicated, in part, on the surprising discovery that administration of expression vectors, in particular adenoviral vectors, can induce beneficial biological responses in the eye, such as the production of gene products that prevent or reduce angiogenesis and sensory cell loss. As used herein, an “adenoviral-responsive gene product” is a gene product encoded by a nucleic acid sequence, the expression of which is upregulated in ocular tissue of the animal, especially mammalian ocular tissue and most preferably human ocular tissue, upon administration of adenovirus or adenoviral vectors. The nucleic acid encoding the adenoviral-responsive gene product can be derived from any source. However, the ocular tissue in which the adenoviral-responsive gene product is upregulated preferably originates from the same species of animal which is treated by the inventive method. In other words, the adenoviral-responsive gene product delivered to the eye of an animal of interest is preferably upregulated in ocular tissue of a biologically matched eye (i.e., upregulated in ocular tissue of the animal of interest or upregulated in an animal of the same species as the animal of interest) when exposed to adenovirus or adenoviral vectors. For example, if the animal of interest is a human, the adenoviral-responsive gene product preferably is human in origin. Ideally, the adenoviral-responsive gene product mediates a desired biological effect in the eye, such as angiogenesis inhibition or protection from sensory cell damage associated with external stimuli or ocular disease.

The adenoviral-responsive gene product is upregulated in ocular tissue in response to administration of adenovirus or adenoviral vectors. Expression is upregulated as compared to expression of the gene product in ocular tissue that has not been administered an adenoviral vector or adenovirus (e.g., untreated ocular tissue or ocular tissue exposed to vehicle). Any level of upregulation, at either the mRNA or protein level, is appropriate in the context of the invention. Gene expression can be analyzed using microarrays and software that quantifies transcription activity, such as Rosetta microarray analysis software. For example, unregulated genes are those genes expressed significantly higher in adenoviral vector-injected eyes compared to eyes with vehicle injection (p<0.01) according to Rosetta error model analysis.

Examples of adenoviral-responsive gene products and nucleic acids encoding adenoviral-responsive gene products from Mus musculus include: hemoglobin alpha, adult chain 1 (Hba-a1), mRNA (GenBank NM_{—}008218); MHC (A.CA/J(H-2K-f) class I antigen (LOC56628), mRNA (NM_{—}019909; hemoglobin, beta adult major chain (Hbb-b1), mRNA (NM_{—}008220); histocompatibility 2, T region locus 22 (H2-T22), mRNA(NM_{—}010397); clone IMAGE:4013674, mRNA (BC025170); H-2 class I histocompatibility antigen, D-B alpha chain precursor (H-2D(B)) (CA575345); histidine rich calcium binding protein (Hrc), mRNA (NM_{—}010473); histocompatibility 2, Q region locus 7 (H2-Q7), mRNA (NM_{—}010394); histocompatibility 2, Q region locus 5 (H2-Q5), mRNA (NM_{—}010393); histocompatibility 2, T region locus 23 (H2-T23), mRNA (NM_{—}010398); histocompatibility 2, D region locus 4 (H2-D4), mRNA (NM_{—}008200); lecithin-retinol acyltransferase (phosphatidylcholine-retinol-O-acyltransferase) (Lrat), mRNA (NM_{—}023624); similar to bone marrow stromal cell antigen 2, clone MGC:28276 IMAGE:4009434, mRNA, complete cds (BC027328); nebulin mRNA, partial cds (AF203898); beta-2 microglobulin (B2m), mRNA (NM_{—}009735); similar to gb:X00172 Mouse H-2K gene fragment encoding a cell surface glycoprotein (AA688940); aminolevulinic acid synthase 2, erythroid (AK077610); histocompatibility 2, Q region locus 1 (H2-Q1), mRNA (NM_{—}010390); partial mRNA for myosin heavy chain IIB (MYHC-IIB gene) (AJ278733); RIKEN cDNA 4930579A11 gene (4930579A11 Rik), mRNA (NM_{—}029478); interferon activated gene 205 (Ifi205), mRNA (NM_{—}172648.1); nuclear-localized inactive X-specific transcript (Xist) mRNA (L04961); perinatal skeletal myosin heavy chain mRNA, 3 end (M12289); partial mRNA for myosin heavy chain IIX (MYHC-IIX gene) (AJ293626); Neutrophil gelatinase-associated lipocalin precursor (NGAL) (P25) (SV-40 induced 24P3 protein) (LIPOCALIN 2) (BC020275); troponin T3, skeletal, fast (Tnnt3), mRNA (NM_{—}011620); interferon regulatory factor 7 (AK079685); guanylate nucleotide binding protein 3 (Gbp3), mRNA (NM_{—}018734); histocompatibility 2, complement component factor B (H2-Bf), mRNA (NM_{—}008198); immunoglobulin-like receptor PIRB5 (6M1) mRNA, complete cds (U96693); troponin C, fast skeletal (Tncs), mRNA (NM_{—}009394); general transcription factor II H, polypeptide 2 (44 kDa subunit) (Gtf2h2), mRNA (NM_{—}022011); NAD(P)(+)—arginine ADP-ribosyltransferase (EC 2.4.2.31) homolog (AK016257); myozenin 1 (Myoz1), mRNA (NM_{—}021508); tropomyosin 1, alpha (Tpm1), mRNA (NM_{—}024427.1); unknown EST (AK010233); allograft inflammatory factor 1 (Aif1), mRNA (NM_{—}019467); CD52 antigen (Cd52), mRNA (NM_{—}013706); myosin, heavy polypeptide 7, cardiac muscle, beta (Myh7), mRNA (NM_{—}080728); myomesin 1 (Myom1), mRNA (NM_{—}010867); RIKEN cDNA 1700025B18 gene (1700025B18Rik), mRNA (NM_{—}025494); proteosome (prosome, macropain) subunit, beta type 8 (large multifunctional protease 7) (Psmb8), mRNA (NM_{—}010724); upregulated during-skeletal muscle growth 4 (Usmg4), mRNA (NM_{—}031401); similar to alpha-interferon inducible protein fragment (AK010014); and peripheral myelin protein, 22 kDa (Pmp22), mRNA (NM_{—}008885).

Given the nucleotide sequence of mouse adenoviral-responsive gene products, the ordinarily skilled artisan can characterize adenoviral-responsive gene products from other species, such as human or rat. A number of human and rat homologs of the adenoviral-responsive gene products recited above have been identified: Novex-3 titin isoform homolog (AK009648); creatine kinase, sarcomeric mitochontrial precursor (EC 2.7.3.2) (S-MTCK) (MIB-CK) (basic-type mitochondrial creatine kinase) homolog (AK009042); HBA,2 hemoglobin, alpha 2 (NP_{—}000508.1); HBA1, hemoglobin, alpha 1 (NP_{—}000549.1); HRC, histidine rich calcium binding protein (NP_{—}002143.1); MR1, major histocompatibility complex (NP_{—}001522.1); HLA-DMB major histocompatibility complex, class II (NP_{—}002109.1); HLA-C, major histocompatibility complex, class I, C(NP_{—}002108.3); HLA-G, histocompatibility antigen, class I, G (NP_{—}002118.1); HLA-F, major histocompatibility complex, class I, F (NP_{—}061823.1); HLA-B, major histocompatibility complex, class I, B (NP_{—}005505.2); HLA-A, major histocompatibility complex, class I, A (NP_{—}002107.3); HLA-E, major histocompatibility complex, class I, E (NP_{—}005507.2); LRAT, lecithin retinol acyltransferase (NP_{—}004735.2); B2M, beta-2-microglobulin (NP_{—}004039.1); VMP 1, likely ortholog of rat vacuole membrane protein (NP_{—}112200.2); TNNT3, troponin T3, skeletal, fast (NP_{—}006748.1); FLJ38822 protein (NP_{—}997281.1); GBP4, guanylate binding protein 4 (NP_{—}443173.2); BF, B-factor, properdin (NP_{—}001701.1); TNNC2 troponin C2, fast (NP_{—}003270.1); GTF2H2, general transcription factor IIH (NP_{—}001506.1); MYOZ1, myozenin 1 (NP_{—}067068.1); AIF1, allograft inflammatory factor 1 (NP_{—}001614.3); MYH7, myosin, heavy polypeptide 7, cardiac muscle (NP_{—}000248.1); ATP6V1C1, ATPase, H+ transporting, lysosomal 42 kDa (NP_{—}001686.1); PSMB8, proteasome (prosome, macropain) subunit (NP_{—}683720.1); and PMP22, peripheral myelin protein 22 (NP_{—}000295.1).

Other adenoviral-responsive gene products produced in ocular tissue can be identified by comparing the nucleic acid sequences of the mouse adenoviral-responsive genes identified herein with, for example, the human genome (or any other genome) using any suitable nucleic acid sequence comparison software, such as BLAST available on the GenBank website (http://www.ncbi.nlm.nih.gov). Homologs of the mouse genes listed above also can be identified using, for example, the Homologene search engine available on the GenBank website. Additional adenovirus-responsive gene products can be characterized using the methods-set forth in Examples 1 and 2. For example, an adenoviral vector, preferably an adenoviral vector which does not contain a therapeutic gene, is administered to an eye, such as a human eye, and tissue samples are collected at various timepoints post-administration. The samples can be subjected to a number of assays, such as rt-PCR and microarray analysis, to determine the gene products which are upregulated in response to vector administration. Ideally, the tissue samples are collected from eyes in which a desired biological response has been detected.

Adenoviral vector administration to the eye upregulates the production of a number of gene products. For example, adenoviral injection into the eye increased expression of apoptosis-related genes, such as p50, caspase-1, and Bcl-2. The adenoviral injection was accompanied by increased levels of photoreceptor cell survival in response to intense light exposure. Thus, factors that impact the NFkB/caspase-1 intracellular signal transduction pathway, including nucleic acids encoding NFkB, can be used in the context of the invention for treating or preventing an ocular-related disorder. Likewise, members of the CC and CXC family of chemokines are upregulated in response to adenoviral vector administration to the eye and are associated with angiogenesis inhibition. CXC chemokines include, for example, PMN chemotactic factors, interleukin-8 (IL-8), ENA-78, GRO-a, GRO-b, and GRO-g. The CC family of chemokines includes, for example, MIP-1, RANTES, MCP-1, MCP-2, MCP-3, and MIP-5. The expression vector of the invention can comprise a nucleic acid sequence encoding any adenovirus-responsive gene product so long as the gene product affects a desired biological response in the eye of the animal of interest, such as inhibiting or reducing angiogenesis or sensory cell loss.

An expression vector encoding an adenoviral-responsive gene product is preferably administered into or adjacent to the eye to treat at least one ocular related disorder. Ocular-related disorders appropriate for treatment using the inventive method include, but are not limited to, diabetic retinopathies, proliferative retinopathies, retinopathy of prematurity, retinal vascular diseases, vascular anomalies, age-related macular degeneration and other acquired disorders, endophthalmitis, infectious diseases, inflammatory diseases, AIDS-related disorders, ocular ischemia syndrome, pregnancy-related disorders, peripheral retinal degenerations, retinal degenerations, toxic retinopathies, cataracts, retinal tumors, corneal neovascularization, choroidal tumors, choroidal disorders, choroidal neovascularization, neovascular glaucoma, vitreous disorders, retinal detachment and proliferative vitreoretinopathy, cyclitis, non-penetrating trauma, penetrating trauma, post-cataract complications, Hippel-Lindau Disease, dry eye, inflammatory optic neuropathies, glaucoma, macular edema, pterygium, iris neovascularization, uveitis, pathologic myopia, surgical-induced disorders, and the like.

The ocular disorder preferably is ocular neovascularization, such as neovascularization of the choroid. The choroid is a thin, vascular membrane located under the retina. Abnormal neovascularization of the choroid results from, for example, photocoagulation, anterior ischemic optic neuropathy, Best's disease, choroidal hemangioma, metallic intraocular foreign body, choroidal nonperfusion, choroidal osteomas, choroidal rupture, bacterial endocarditis, choroideremia, chronic retinal detachment, drusen, deposit of metabolic waste material, endogenous Candida endophthalmitis, neovascularization at ora serrata, operating microscope burn, punctate inner choroidopathy, radiation retinopathy, retinal cryoinjury, retinitis pigmentosa, retinochoroidal coloboma, rubella, subretinal fluid drainage, tilted disc syndrome, Taxoplasma retinochoroiditis, tuberculosis, and the like.

Neovascularization of the cornea also is appropriate for treatment by the method of the invention. The cornea is a projecting, transparent section of the fibrous tunic, the outer most layer of the eye. The outermost layer of the cornea contacts the conjunctiva, while the innermost layer comprises the endothelium of the anterior chamber. Corneal neovascularization stems from, for example, ocular injury, surgery, infection, improper wearing of contact lenses, and diseases such as, for example, corneal dystrophies.

Alternatively, the ocular neovascularization is neovascularization of the retina. Retinal neovascularization is an indication associated with numerous ocular diseases and disorders, many of which are named above. Preferably, the neovascularization of the retina treated in accordance with the inventive method is associated with diabetic retinopathy. Common causes of retinal neovascularization include ischemia, viral infection, and retinal damage. Neovascularization of the retina can lead to macular edema, subretinal discoloration, scarring, hemorrhaging, and the like. Complications associated with retina neovascularization stem from growth, breakage, and leakage of newly formed blood vessels. Vision is impaired as blood fills the vitreous cavity and is not efficiently removed. Not only is the passage of light impeded, but an inflammatory response to the excess blood and metabolites can cause further damage to ocular tissue. In addition, the new vessels form fibrous scar tissue, which, over time, will disturb the retina causing retinal tears and detachment.

The ocular disorder can be age-related macular degeneration, which can involve both exudative (neovascular) and atrophic complications. Exudative complications include, for example, disciform scars (i.e., scarring involving fibrous elements) and neovascularization. Atrophic complications include, for instance, the formation of drusen and basal laminar deposits, irregularity of retinal pigmentation, and accumulation of lipofuscin granules. The ocular disorder also can be ocular edema (e.g., retinal edema or macular edema).

By “prophylactic” is meant the protection, in whole or in part, against ocular-related disorders, in particular ocular neovascularization or age-related macular degeneration. By “therapeutic” is meant the amelioration of the ocular-related disorder, itself, and the protection, in whole or in part, against further ocular-related disease, in particular ocular neovascularization or age-related macular degeneration. One of ordinary skill in the art will appreciate that any degree of protection from, or amelioration of, an ocular-related disorder is beneficial to a patient. The inventive method also can be useful for protecting against or reducing the effects of symptoms of an ocular-related disorder. For example, the inventive method can inhibit angiogenesis in the eye (i.e., prevent the formation of new blood vessels in the eye) or reduce the level of angiogenesis (i.e., promote the destruction of newly formed blood vessels). Likewise, the inventive method can inhibit or reduce sensory cell loss, such as photoreceptor cell loss, in any layer of the eye.

The inventive method can be used to treat both acute and persistent, progressive ocular-related disorders. For acute ailments, the expression vector can be administered using a single application or multiple applications within a short time period. For persistent ocular-related disorders, such as age-related macular degeneration, diabetic retinopathy or prolonged deterioration of retinal pigment epithelial cells, numerous applications of the expression vector may be necessary to realize a desired biological effect.

If desired, the inventive method can further comprise inducing a stress response in the eye prior to administering an expression vector. It has surprisingly been determined that treating an eye with, for example, photocoagulation or photodynamic therapy increases in ocular cells the expression of cell surface molecules which facilitate transduction by expression vectors and cell adhesion. In particular, the cell surface molecules most recognized as mediating adenoviral infection, coxsackievirus and adenovirus receptor (CAR) and integrins (e.g., integrins β3 and β5), are upregulated in the retina and choroid following, for example, photocoagulation therapy. Upregulation of CAR and integrins allows transduction of a greater number of host cells with an adenoviral vector encoding a gene product as compared to adenoviral transduction efficiency without induction of the stress response (e.g., laser therapy). Accordingly, the level of transduction of host cells by the expression vector is enhanced as compared to the level of transduction of host cells by the expression vector in the absence of inducing a stress response in the eye. In some instances, inducing a stress response prior to vector administration allows delivery of a smaller dose of expression vector than previously thought possible to achieve a desired biological response.

In addition to increasing transduction of host cells by the expression vector, inducing a stress response in the eye prior to administering the expression vector enhances and prolongs expression of the nucleic acid sequence of the expression vector compared to expression of the nucleic acid sequence in the absence of the stress response. Ideally, expression of the nucleic acid sequence in the context of the inventive method is enhanced compared to expression of the nucleic acid sequence in the absence of inducing the stress response in the eye (but under otherwise similar conditions) for at least one day (preferably at least 3 days (e.g., 1, 2, or 3 days) or at least five days) following administration of the expression vector. More preferably, expression of the nucleic acid sequence is enhanced for at least 7 days (e.g., at least 14 days or at least 21 days) post-administration of the expression vector as compared to expression of the nucleic acid sequence in the absence of the stress response (e.g., in the absence of photocoagulation therapy) at the same timepoint. Even more preferably, expression of the nucleic acid sequence is enhanced as compared to expression of the nucleic acid sequence in the absence of inducing a stress response for at least 28 days (e.g., at least 60 days or at least 90 days) post-administration of the expression vector to the eye.

By “enhanced” expression is meant any increase in transcription compared to transcription (i.e., gene or nucleic acid sequence expression) which occurs in the absence of inducing the stress response. Any increase in expression of the nucleic acid sequence is appropriate in the context of the invention. For example, enhanced expression of the nucleic acid sequence can be a 2-, 3-, 5-, 10-, 20-, or 50-fold increase in expression as compared to the level of gene expression which occurs under similar conditions but in the absence of inducing the stress response in the eye. The enhanced expression (transcription) can result in increased levels of RNA transcript, increased protein production, and/or an enhancement in detectable gene product activity, all of which can be detected using routine laboratory techniques.

A stress response can be induced in the eye by exposure to heat using, for example, lasers in photodynamic therapy, exposure to cold, exposure to light, exposure to radiation (e.g., X-rays), exposure to microwaves, exposure to ultrasound, or physical trauma, all of which can alter the ocular cellular environment to enhance transcription. Desirably, inducing a stress response in the eye comprises applying photodynamic therapy or photocoagulation therapy to the eye.

In addition, given that the administration of an adenoviral vector upregulates adenoviral-responsive gene products having a desirable biological effect in the eye, an adenoviral vector, such as an adenoviral vector that does not comprise a therapeutic transgene (an “AdNull” vector), can be administered to an eye to upregulate transcription of therapeutic adenoviral-responsive gene products. The administration of an AdNull vector to the eye can achieve a biologically beneficial response in the eye via upregulation of nucleic acids encoding adenoviral-responsive gene products.

One of ordinary skill in the art will appreciate that any of a number of expression vectors known in the art are suitable for use in the inventive method. Examples of suitable expression vectors include, for instance, plasmids, plasmid-liposome complexes, and viral vectors, e.g., parvoviral-based vectors (i.e., adeno-associated virus (AAV)-based vectors), retroviral vectors, herpes simplex virus (HSV)-based vectors, AAV-adenoviral chimeric vectors, and adenovirus-based vectors. Any of these expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

Preferably, the expression vector is a viral vector. More preferably, the expression vector is an adenoviral vector, e.g., a human adenoviral vector. In the context of the invention, the adenoviral vector can be derived from any serotype of adenovirus. Adenoviral stocks that can be employed as a source of adenovirus can be amplified from the adenoviral serotypes 1 through 51, which are currently available from the American Type Culture Collection (ATCC, Manassas, Va.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), an unclassified serogroup (e.g., serotypes 49 and 51), or any other adenoviral serotype. Preferably, however, an adenovirus is of serotype 2, 5, or 9. However, non-group C adenoviruses can be used to prepare replication-deficient adenoviral gene transfer vectors for delivery of gene products to host cells, such as ocular cells. Preferred adenoviruses used in the construction of non-group C adenoviral gene transfer vectors include Ad12 (group A), Ad7 and Ad35 (group B), Ad30 and Ad36 (group D), Ad4 (group E), and Ad41 (group F). Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561 and International Patent Applications WO 97/12986 and WO 98/53087.

The adenoviral vector is preferably deficient in at least one gene function required for viral replication, thereby resulting in a “replication-deficient” adenoviral vector. By “replication-deficient” is meant that the adenoviral vector comprises an adenoviral genome that lacks at least one replication-essential gene function (i.e., such that the adenoviral vector does not replicate in typical host cells, especially those in the human patient that could be infected by the adenoviral vector in the course of treatment in accordance with the invention). A deficiency in a gene, gene function, or gene or genomic region, as used herein, is defined as a deletion of sufficient genetic material of the viral genome to impair or obliterate the function of the gene (e.g., such that the function of the gene product is reduced by at least about 2-fold, 5-fold, 10-fold, 20-fold, 30-fold, or 50-fold) whose nucleic acid sequence was deleted in whole or in part. Deletion of an entire gene region often is not required for disruption of a replication-essential gene function. However, for the purpose of providing sufficient space in the adenoviral genome for one or more transgenes, removal of a majority of a gene region may be desirable. Replication-essential gene functions are those gene functions that are required for replication (e.g., propagation) and are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA-1 and/or VA-RNA-2). More preferably, the replication-deficient adenoviral vector comprises an adenoviral genome deficient in at least one replication-essential gene function of one or more regions of the adenoviral genome. In this respect, the adenoviral vector desirably is deficient in at least one essential gene function of the E1 region of the adenoviral genome required for viral replication. In addition to a deficiency in the E1 region, the recombinant adenovirus can also have a mutation in the major late promoter (MLP). The mutation in the MLP can be in any of the MLP control elements such that it alters the responsiveness of the promoter, as discussed in International Patent Application WO 00/00628. More preferably, the vector is deficient in at least one essential gene function of the E1 region and at least part of the E3 region (e.g., an Xba I deletion of the E3 region). With respect to the E1 region, the adenoviral vector can be deficient in at least part of the E1a region and at least part of the E1b region. For example, the adenoviral vector can comprise a deletion of the entire E1 region and part of the E3 region of the adenoviral genome (i.e., nucleotides 355 to 3,511 and 28,593 to 30,470). When the adenoviral vector is E1-deficient, the adenoviral vector genome can comprise a deletion beginning at any nucleotide between nucleotides 335 to 375 (e.g., nucleotide 356) and ending at any nucleotide between nucleotides 3,310 to 3,350 (e.g., nucleotide 3,329) or even ending at any nucleotide between 3,490 and 3,530 (e.g., nucleotide 3,510) (based on the adenovirus serotype 5 genome). When E3-deficient, the adenoviral vector genome can comprise a deletion beginning at any nucleotide between nucleotides 28,575 to 29,615 (e.g., nucleotide 28,593) and ending at any nucleotide between nucleotides 30,450 to 30,490 (e.g., nucleotide 30,470) (based on the adenovirus serotype 5 genome). Therefore, for example, a singly-deficient adenoviral vector can be deleted of approximately nucleotides 356 to 3,329 and 28,594 to 30,469 (based on the adenovirus serotype 5 genome). Alternatively, the adenoviral vector genome can be deleted of approximately nucleotides 356 to 3,510 and 28,593 to 30,470 (based on the adenovirus serotype 5 genome). The endpoints defining the deleted nucleotide portions can be difficult to precisely determine and typically will not significantly affect the nature of the adenoviral vector, i.e., each of the aforementioned nucleotide numbers can be +/−1, 2, 3, 4, 5, or even 10 or 20 nucleotides.

Preferably, the adenoviral vector is “multiply deficient,” meaning that the adenoviral vector is deficient in one or more essential gene functions required for viral replication in each of two or more regions. For example, the aforementioned E1-deficient or E1-, E3-deficient adenoviral vectors can be further deficient in at least one essential gene function of the E4 region. Adenoviral vectors deleted of the entire E4 region can elicit lower host immune responses. When E4-deficient, the adenoviral vector genome can comprise a deletion of, for example, nucleotides 32,826 to 35,561 (based on the adenovirus serotype 5 genome), optionally in addition to deletions in the E1 region (e.g., nucleotides 356 to 3,329 or nucleotides 356 to 3,510) and/or deletions in the E3 region (e.g., nucleotides 28,594 to 30,469 or nucleotides 28,593 to 30,470).

Alternatively, the adenoviral vector lacks all or part of the E1 region and all or part of the E2 region (e.g., the E2A region). When E2A-deficient, the adenoviral vector genome can comprise a deletion beginning at any nucleotide between nucleotides 22,425 to 22,465 (e.g., nucleotide 22,443) and ending at any nucleotide between nucleotides 24,010 to 24,050 (e.g., nucleotide 24,032) (based on the adenovirus serotype 5 genome). However, adenoviral vectors lacking all or part of the E1 region, all or part of the E2 region, and all or part of the E3 region also are contemplated herein. In one embodiment, the adenoviral vector lacks all or part of the E1 region, all or part of the E2 region, all or part of the E3 region, and all or part of the E4 region. Suitable replication-deficient adenoviral vectors are disclosed in U.S. Pat. Nos. 5,851,806 and 5,994,106 and International Patent Applications WO 95/34671 and WO 97/21826. For example, suitable replication-deficient adenoviral vectors include those with at least a partial deletion of the E1a region, at least a partial deletion of the E1b region, at least a partial deletion of the E2a region, and at least a partial deletion of the E3 region. Alternatively, the replication-deficient adenoviral vector can have at least a partial deletion of the E1 region, at least a partial deletion of the E3 region, and at least a partial deletion of the E4 region. Alternatively or in addition, other regions of the adenoviral genome also can be deleted such as the VAI gene and VAII gene as described in International Patent Application No. PCT/US02/29111. Multiply-deficient viral vectors are particularly useful in that such vectors can accept large inserts of exogenous DNA. Indeed, adenoviral amplicons, an example of a multiply-deficient adenoviral vector which comprises only those genomic sequences required for packaging and replication of the viral genome, can accept inserts of approximately 36 kb.

If the adenoviral vector of the invention is deficient in a replication-essential gene function of the E2A region, the vector preferably does not comprise a complete deletion of the E2A region, which deletion preferably is less than about 230 base pairs in length. Generally, the E2A region of the adenovirus codes for a DBP (DNA binding protein), a polypeptide required for DNA replication. DBP is composed of 473 to 529 amino acids depending on the viral serotype. It is believed that DBP is an asymmetric protein that exists as a prolate ellipsoid consisting of a globular Ct with an extended Nt domain. Studies indicate that the Ct domain is responsible for DBP's ability to bind to nucleic acids, bind to zinc, and function in DNA synthesis at the level of DNA chain elongation. However, the Nt domain is believed to function in late gene expression at both transcriptional and post-transcriptional levels, is responsible for efficient nuclear localization of the protein, and also may be involved in enhancement of its own expression. Deletions in the Nt domain between amino acids 2 to 38 have indicated that this region is important for DBP function (Brough et al., Virology, 196, 269-281 (1993)). While deletions in the E2A region coding for the Ct region of the DBP have no effect on viral replication, deletions in the E2A region which code for amino acids 2 to 38 of the Nt domain of the DBP impair viral replication. It is preferable that any multiply replication-deficient adenoviral vector contains this portion of the E2A region of the adenoviral genome. In particular, for example, the desired portion of the E2A region to be retained is that portion of the E2A region of the adenoviral genome which is defined by the 5′ end of the E2A region, specifically positions Ad5(23816) to Ad5(24032) of the E2A region of the adenoviral genome of serotype Ad5. This portion of the adenoviral genome desirably is included in the adenoviral vector because it is not complemented in current E2A cell lines so as to provide the desired level of viral propagation.

Therefore, in a preferred embodiment, the expression vector of the inventive method is a multiply-deficient adenoviral vector lacking all or part of the E1 region, all or part of the E3 region, all or part of the E4 region, and, optionally, all or part of the E2 region. In this regard, it has been observed that an at least E4-deficient adenoviral vector expresses a transgene at high levels for a limited amount of time in vivo and that persistence of expression of a transgene in an at least E4-deficient adenoviral vector can be modulated through the action of a trans-acting factor, such as HSV ICP0, Ad pTP, CMV-IE2, CMV-IE86, HIV tat, HTLV-tax, HBV-X, AAV Rep 78, the cellular factor from the U205 osteosarcoma cell line that functions like HSV ICP0, or the cellular factor in PC12 cells that is induced by nerve growth factor, among others. In view of the above, a nucleic acid sequence encoding a trans-acting factor that modulates the persistence of expression of the nucleic acid sequence encoding the gene product can be administered. Use of trans-acting factors in combination with replication deficient adenoviral vectors is further described in U.S. Pat. Nos. 6,225,113, 6,660,521, and 6,649,373 and International Patent Application WO 00/34496.

It should be appreciated that the deletion of different regions of the adenoviral vector can alter the immune response of the mammal. In particular, deletion of different regions can reduce the inflammatory response generated by the adenoviral vector. Furthermore, the adenoviral vector's coat protein can be modified so as to decrease the adenoviral vector's ability or inability to be recognized by a neutralizing antibody directed against the wild-type coat protein, as described in International Patent Application WO 98/40509. Such modifications are useful for long-term treatment of persistent ocular disorders.

The adenoviral vector, when multiply replication-deficient, especially in replication-essential gene functions of the E1 and E4 regions, preferably includes a spacer element to provide viral growth in a complementing cell line similar to that achieved by singly replication-deficient adenoviral vectors, particularly an adenoviral vector comprising a deficiency in the E1 region. In a preferred E4-deficient adenoviral vector of the invention wherein the L5 fiber region is retained, the spacer is desirably located between the L5 fiber region and the right-side ITR. More preferably in such an adenoviral vector, the E4 polyadenylation sequence alone or, most preferably, in combination with another sequence exists between the L5 fiber region and the right-side ITR, so as to sufficiently separate the retained L5 fiber region from the right-side ITR, such that viral production of such a vector approaches that of a singly replication-deficient adenoviral vector, particularly a singly replication-deficient E1 deficient adenoviral vector.

The spacer element can contain any sequence or sequences which are of a desired length, such as sequences at least about 15 base pairs (e.g., between about 15 base pairs and about 12,000 base pairs), preferably about 100 base pairs to about 10,000 base pairs, more preferably about 500 base pairs to about 8,000 base pairs, even more preferably about 1,500 base pairs to about 6,000 base pairs, and most preferably about 2,000 to about 3,000 base pairs in length. The spacer element sequence can be coding or non-coding and native or non-native with respect to the adenoviral genome, but does not restore the replication-essential function to the deficient region. The spacer can also contain a promoter-variable expression cassette. More preferably, the spacer comprises an additional polyadenylation sequence and/or a passenger gene. Preferably, in the case of a spacer inserted into a region deficient for E4, both the E4 polyadenylation sequence and the E4 promoter of the adenoviral genome or any other (cellular or viral) promoter remain in the vector. The spacer is located between the E4 polyadenylation site and the E4 promoter, or, if the E4 promoter is not present in the vector, the spacer is proximal to the right-side ITR. The spacer can comprise any suitable polyadenylation sequence. Examples of suitable polyadenylation sequences include synthetic optimized sequences, BGH (Bovine Growth Hormone), polyoma virus, TK (Thymidine Kinase), EBV (Epstein Barr Virus) and the papillomaviruses, including human papillomaviruses and BPV (Bovine Papilloma Virus). Preferably, particularly in the E4 deficient region, the spacer includes an SV40 polyadenylation sequence. The SV40 polyadenylation sequence allows for higher virus production levels of multiply replication deficient adenoviral vectors. In the absence of a spacer, production of fiber protein and/or viral growth of the multiply replication-deficient adenoviral vector is reduced by comparison to that of a singly replication-deficient adenoviral vector. However, inclusion of the spacer in at least one of the deficient adenoviral regions, preferably the E4 region, can counteract this decrease in fiber protein production and viral growth. Ideally, the spacer is composed of the glucuronidase gene. The use of a spacer in an adenoviral vector is further described in, for example, U.S. Pat. No. 5,851,806 and International Patent Application WO 97/21826.

Desirably, the adenoviral vector requires, at most, complementation of replication-essential gene functions of the E1, E2A, and/or E4 regions of the adenoviral genome for replication (i.e., propagation). However, the adenoviral genome can be modified to disrupt one or more replication-essential gene functions as desired by the practitioner, so long as the adenoviral vector remains deficient and can be propagated using, for example, complementing cells and/or exogenous DNA (e.g., helper adenovirus) encoding the disrupted replication-essential gene functions. In this respect, the adenoviral vector can be deficient in replication-essential gene functions of only the early regions of the adenoviral genome, only the late regions of the adenoviral genome, and both the early and late regions of the adenoviral genome. The adenoviral vector also can have essentially the entire adenoviral genome removed, in which case it is preferred that at least the viral inverted terminal repeats (ITRs) and a packaging signal are left intact (i.e., an adenoviral amplicon). Suitable replication-deficient adenoviral vectors, including multiply replication-deficient adenoviral vectors, are disclosed in U.S. Pat. Nos. 5,837,511; 5,851,806; 5,994,106; and 6,579,522; U.S. Published Patent Applications 2001/0043922 A1, 2002/0004040 A1, 2002/0031831 A1, and 2002/0110545 A1, and International Patent Applications WO 95/34671, WO 97/12986, and WO 97/21826.

Ideally, when the expression vector is an adenoviral vector, the adenoviral vector is administered in a pharmaceutical composition virtually free of replication-competent adenovirus (RCA) contamination (e.g., the pharmaceutical composition comprises less than about 1% of RCA contamination). Most desirably, the pharmaceutical composition is RCA-free. Adenoviral vector compositions and stocks that are RCA-free are described in U.S. Pat. Nos. 5,944,106 and 6,482,616, U.S. Published Patent Application 2002/0110545 A1, and International Patent Application WO 95/34671. Ideally, the pharmaceutical composition also is free of E1-revertants when the adenoviral vector is E1-deficient in combination with deficiencies in other replication-essential gene functions of another region of the adenoviral genome, as further described in International Patent Application WO 03/040314.

In addition to modification (e.g., deletion, mutation, or replacement) of adenoviral sequences encoding replication-essential gene functions, the adenoviral genome can contain benign or non-lethal modifications, i.e., modifications which do not render the adenovirus replication-deficient, or, desirably, do not adversely affect viral functioning and/or production of viral proteins, even if such modifications are in regions of the adenoviral genome that otherwise contain replication-essential gene functions. Such modifications commonly result from DNA manipulation or serve to facilitate expression vector construction. For example, it can be advantageous to remove or introduce restriction enzyme sites in the adenoviral genome. Such benign mutations often have no detectable adverse effect on viral functioning. For example, the adenoviral vector can comprise a deletion of nucleotides 10,594 and 10,595 (based on the adenoviral serotype 5 genome), which are associated with VA-RNA-1 transcription, but the deletion of which does not prohibit production of VA-RNA-1.

Similarly, the coat protein of a viral vector, preferably an adenoviral vector, can be manipulated to alter the binding specificity or recognition of a virus for a viral receptor on a potential host cell. For adenovirus, such manipulations can include deletion of regions of the fiber, penton, or hexon, insertions of various native or non-native ligands into portions of the coat protein, and the like. Manipulation of the coat protein can broaden the range of cells infected by a viral vector or enable targeting of a viral vector to a specific cell type.

For example, in one embodiment, the expression vector is an adenoviral vector comprising a chimeric coat protein (e.g., a fiber, hexon pIX, pIIIa, or penton protein), which differs from the wild-type (i.e., native) coat protein by the introduction of a normative amino acid sequence, preferably at or near the carboxyl terminus. Preferably, the normative amino acid sequence is inserted into or in place of an internal coat protein sequence. One of ordinary skill in the art will understand that the normative amino acid sequence can be inserted within the internal coat protein sequence or at the end of the internal coat protein sequence. The resultant chimeric viral coat protein is able to direct entry into cells of the viral, i.e., adenoviral, vector comprising the coat protein that is more efficient than entry into cells of a vector that is identical except for comprising a wild-type viral coat protein rather than the chimeric viral coat protein. Preferably, the chimeric virus coat protein binds a novel endogenous binding site present on the cell surface that is not recognized, or is poorly recognized, by a vector comprising a wild-type coat protein. One direct result of this increased efficiency of entry is that the virus, preferably, the adenovirus, can bind to and enter numerous cell types which a virus comprising wild-type coat protein typically cannot enter or can enter with only a low efficiency.

In another embodiment of the invention, the expression vector is a viral vector comprising a chimeric virus coat protein not selective for a specific type of eukaryotic cell. The chimeric coat protein differs from the wild-type coat protein by an insertion of a nonnative amino acid sequence into or in place of an internal coat protein sequence. In this embodiment, the chimeric virus coat protein efficiently binds to a broader range of eukaryotic cells than a wild-type virus coat, such as described in International Patent Application WO 97/20051.

Specificity of binding of an adenovirus to a given cell can also be adjusted by use of an adenovirus comprising a short-shafted adenoviral fiber gene, as discussed in U.S. Pat. No. 5,962,311. Use of an adenovirus comprising a short-shafted adenoviral fiber gene reduces the level or efficiency of adenoviral fiber binding to its cell-surface receptor and increases adenoviral penton base binding to its cell-surface receptor, thereby increasing the specificity of binding of the adenovirus to a given cell. Alternatively, use of an adenovirus comprising a short-shafted fiber enables targeting of the adenovirus to a desired cell-surface receptor by the introduction of a normative amino acid sequence either into the penton base or the fiber knob.

Of course, the ability of a viral vector to recognize a potential host cell can be modulated without genetic manipulation of the coat protein. For instance, complexing an adenovirus with a bispecific molecule comprising a penton base-binding domain and a domain that selectively binds a particular cell surface binding site enables one of ordinary skill in the art to target the vector to a particular cell type.

Suitable modifications to a viral vector, specifically an adenoviral vector, are described in U.S. Pat. Nos. 5,543,328; 5,559,099; 5,712,136; 5,731,190; 5,756,086; 5,770,442; 5,846,782; 5,871,727; 5,885,808; 5,922,315; 5,962,311; 5,965,541; 6,057,155; 6,127,525; 6,153,435; 6,329,190; 6,455,314; and 6,465,253; U.S. Patent Application Publications 2001/0047081 A1, 2002/0099024 A1, and 2002/0151027 A1, and International Patent Applications WO 95/02697, WO 95/16772, WO 95/34671, WO 96/07734, WO 96/22378, WO 96/26281, WO 97/20051, WO 98/07865, WO 98/07877, WO 98/40509, WO 98/54346, WO 00/15823, WO 01/58940, and WO 01/92549. Similarly, it will be appreciated that numerous expression vectors are available commercially. Construction of expression vectors is well understood in the art. Adenoviral vectors can be constructed and/or purified using methods known in the art (e.g., using complementing cell lines, such as the 293 cell line, Per.C6 cell line, or 293-ORF6 cell line) and methods set forth, for example, in U.S. Pat. Nos. 5,965,358; 5,994,128; 6,033,908; 6,168,941; 6,329,200; 6,383,795; 6,440,728; 6,447,995; and 6,475,757; U.S. Patent Application Publication 2002/0034735 A1, and International Patent Applications WO 98/53087, WO 98/56937, WO 99/15686, WO 99/54441, WO 00/12765, WO 01/77304, and WO 02/29388, as well as the other references identified herein. Adeno-associated viral vectors can be constructed and/or purified using the methods set forth, for example, in U.S. Pat. No. 4,797,368 and Laughlin et al., Gene, 23, 65-73 (1983).

The selection of expression vector for use in the inventive method will depend on a variety of factors such as, for example, the host, immunogenicity of the vector, the desired duration of protein production, and the like. As each type of expression vector has distinct properties, a researcher has the freedom to tailor the inventive method to any particular situation. Moreover, more than one type of expression vector can be used to deliver the gene product to the host cell.

The nucleic acid sequence in the expression vector is desirably present as part of an expression cassette, i.e., a particular nucleotide sequence that possesses functions which facilitate subcloning and recovery of a nucleic acid sequence (e.g., one or more restriction sites) or expression of a nucleic acid sequence (e.g., polyadenylation or splice sites). When the expression vector is an adenoviral vector, the nucleic acid sequence is preferably located in the E1 region (e.g., replaces the E1 region in whole or in part) of the adenoviral genome. For example, the E1 region can be replaced by a promoter-variable expression cassette comprising the nucleic acid sequence(s). The expression cassette is preferably inserted in a 3′-5′ orientation, e.g., oriented such that the direction of transcription of the expression cassette is opposite that of the surrounding adjacent adenoviral genome. In addition to the expression cassette comprising the nucleic acid sequence(s), the adenoviral vector can comprise other expression cassettes containing nucleic acid sequences encoding other products, which cassettes can replace any of the deleted regions of the adenoviral genome. The insertion of an expression cassette into the adenoviral genome (e.g., into the E1 region of the genome) can be facilitated by known methods, for example, by the introduction of a unique restriction site at a given position of the adenoviral genome. As set forth above, preferably all or part of the E3 region of the adenoviral vector also is deleted.

Nucleic acid sequences are routinely operably linked to regulatory sequences necessary for expression, i.e., a promoter. A “promoter” is a DNA sequence that directs the binding of RNA polymerase and thereby promotes RNA synthesis. A nucleic acid sequence is “operably linked” to a promoter when the promoter is capable of directing transcription of that nucleic acid sequence. A promoter can be native or non-native to the nucleic acid sequence to which it is operably linked.

Any promoter (i.e., whether isolated from nature or produced by recombinant DNA or synthetic techniques) can be used in connection with the invention to provide for transcription of the nucleic acid sequence. The promoter preferably is capable of directing transcription in a eukaryotic (desirably mammalian) cell. The functioning of the promoter can be altered by the presence of one or more enhancers and/or silencers present on the vector. “Enhancers” are cis-acting elements of DNA that stimulate or inhibit transcription of adjacent genes. An enhancer that inhibits transcription also is termed a “silencer.” Enhancers differ from DNA-binding sites for sequence-specific DNA binding proteins found only in the promoter (which also are termed “promoter elements”) in that enhancers can function in either orientation, and over distances of up to several kilobase pairs (kb), even from a position downstream of a transcribed region.

Promoter regions can vary in length and sequence and can further encompass one or more DNA binding sites for sequence-specific DNA binding proteins and/or an enhancer or silencer. Enhancers and/or silencers can similarly be present on a nucleic acid sequence outside of the promoter per se. Desirably, a cellular or viral enhancer, such as the cytomegalovirus (CMV) immediate-early enhancer, is positioned in the proximity of the promoter to enhance promoter activity. In addition, splice acceptor and donor sites can be present on a nucleic acid sequence to enhance transcription.

The invention preferentially employs a viral promoter. Suitable viral promoters are known in the art and include, for instance, cytomegalovirus (CMV) promoters, such as the CMV immediate-early promoter, promoters derived from human immunodeficiency virus (HIV), such as the HIV long terminal repeat promoter, Rous sarcoma virus (RSV) promoters, such as the RSV long terminal repeat, mouse mammary tumor virus (MMTV) promoters, HSV promoters, such as the Lap2 promoter or the herpes thymidine kinase promoter (Wagner et al., Proc. Natl. Acad. Sci., 78, 144-145 (1981)), promoters derived from SV40 or Epstein Barr virus, an adeno-associated viral promoter, such as the p5 promoter, and the like. Preferably, the viral promoter is an adenoviral promoter, such as the Ad2 or Ad5 major late promoter and tripartite leader, a CMV promoter, or an RSV promoter.

Alternatively, the invention employs a cellular promoter, i.e., a promoter that drives expression of a cellular protein. Preferred cellular promoters for use in the invention will depend on the desired expression profile to produce the therapeutic agent(s). In one aspect, the cellular promoter is preferably a constitutive promoter that works in a variety of cell types, such as cells associated with the eye. Suitable constitutive promoters can drive expression of genes encoding transcription factors, housekeeping genes, or structural genes common to eukaryotic cells. For example, the Ying Yang 1 (YY1) transcription factor (also referred to as NMP-1, NF-E1, and UCRBP) is a ubiquitous nuclear transcription factor that is an intrinsic component of the nuclear matrix (Guo et al., PNAS, 92, 10526-10530 (1995)). While the promoters described herein are considered as constitutive promoters, it is understood in the art that constitutive promoters can be upregulated. Promoter analysis shows that the elements critical for basal transcription reside from −277 to +475 of the YY1 gene relative to the transcription start site from the promoter, and include a TATA and CCAAT box. JEM-1 (also known as HGMW and BLZF-1) also is a ubiquitous nuclear transcription factor identified in normal and tumorous tissues (Tong et al., Leukemia, 12(11), 1733-1740 (1998), and Tong et al., Genomics, 69(3), 380-390 (2000)). JEM-1 is involved in cellular growth control and maturation, and can be upregulated by retinoic acids. Sequences responsible for maximal activity of the JEM-1 promoter has been located at −432 to +101 of the JEM-1 gene relative the transcription start site of the promoter. Unlike the YY1 promoter, the JEM-1 promoter does not comprise a TATA box. The ubiquitin promoter, specifically UbC, is a strong constitutively active promoter functional in several species. The UbC promoter is further characterized in Marinovic et al., J. Biol. Chem., 277(19), 16673-16681(2002).

Many of the above-described promoters are constitutive promoters. Instead of being a constitutive promoter, the promoter can be an inducible promoter, i.e., a promoter that is up- and/or down-regulated in response to appropriate signals. For instance, the regulatory sequences can comprise a hypoxia driven promoter, which is active when an ocular disorder is associated with hypoxia. Other examples of suitable inducible promoter systems include, but are not limited to, the IL-8 promoter, the metallothionine inducible promoter system, the bacterial lacZYA expression system, the tetracycline expression system, and the T7 polymerase system. Further, promoters that are selectively activated at different developmental stages (e.g., globin genes are differentially transcribed from globin-associated promoters in embryos and adults) can be employed. The promoter sequence that regulates expression of the nucleic acid sequence can contain at least one heterologous regulatory sequence responsive to regulation by an exogenous agent. The regulatory sequences are preferably responsive to exogenous agents such as, but not limited to, drugs, hormones, or other gene products (ideally gene products produced in the eye). For example, the regulatory sequences, e.g., promoter, preferably are responsive to glucocorticoid receptor-hormone complexes, which, in turn, enhance the level of transcription of a therapeutic gene or a therapeutic fragment thereof.

The regulatory sequences can comprise a tissue-specific promoter, i.e., a promoter that is preferentially activated in a given tissue and results in expression of a gene product in the tissue where activated. A tissue-specific promoter suitable for use in the invention can be chosen by the ordinarily skilled artisan based upon the target tissue or cell-type. Preferred tissue-specific promoters for use in the inventive method are specific to ocular tissue, such as a rhodopsin promoter. Examples of rhodopsin promoters include, but are not limited to, a GNAT cone transducing alpha-subunit gene promoter or an interphotoreceptor retinoid binding protein promoter.

Also preferably, the expression vector comprises a nucleic acid encoding a cis-acting factor, wherein the cis-acting factor modulates the expression of the nucleic acid sequence. Preferably, the cis-acting factor comprises matrix attachment region (MAR) sequences (e.g., immunoglobulin heavy chain (Jenunwin et al., Nature, 385(16), 269 (1997)), apolipoprotein B, or locus control region (LCR) sequences, among others. MAR sequences have been characterized as DNA sequences that associate with the nuclear matrix after a combination of nuclease digestion and extraction (Bode et al., Science, 255(5041), 195-197 (1992)). MAR sequences are often associated with enhancer-type regulatory regions, and, when integrated into genomic DNA, MAR sequences augment transcriptional activity of adjacent nucleotide sequences. It has been postulated that MAR sequences play a role in controlling the topological state of chromatin structures, thereby facilitating the formation of transcriptionally-active complexes. Similarly, it is believed LCR sequences function to establish and/or maintain domains permissive for transcription. Many LCR sequences give tissue specific expression-of-associated nucleic acid sequences. Addition of MAR or LCR sequences to the expression vector can further enhance expression of the nucleic acid sequence.

To optimize production of the adenoviral-responsive gene product, preferably the nucleic acid sequence further comprises a polyadenylation site following the coding region of the nucleic acid sequence. Also, preferably all the proper transcription signals (and translation signals, where appropriate) will be correctly arranged such that the nucleic acid sequence will be properly expressed in the cells into which it is introduced. If desired, the nucleic acid sequence also can incorporate splice sites (i.e., splice acceptor and splice donor sites) to facilitate mRNA production. Moreover, if the nucleic acid sequence encodes an adenoviral-responsive gene product, which is a processed or secreted protein or acts intracellularly, preferably the nucleic acid sequence further comprises the appropriate sequences for processing, secretion, intracellular localization, and the like.

In addition to the nucleic acid encoding the adenoviral-responsive gene product, the expression vector of the inventive method can comprise another nucleic acid sequence that encodes one or more other gene products. The other gene products can be a protein or RNA useful in methods of treatment, diagnostic methods, or useful in ocular-related research. In one embodiment, the gene product is a therapeutic gene product (i.e., a gene product that achieves a beneficial biological effect in a patient). Suitable gene products include, but are not limited to, cytokines, enzymes, inhibitors of angiogenesis, neurotrophic factors, antibodies, and biologically-active fragments of any of the foregoing. Alternatively, the method of the invention can comprise administering one or more other expression vectors, such as adenoviral vectors, which encode any of the aforementioned gene products.

Preferably, the inventive method comprises administering to a subject a nucleic acid encoding an inhibitor of angiogenesis. The nucleic acid sequence can encode multiple inhibitors of angiogenesis. By “inhibitor of angiogenesis” is meant any factor that prevents or ameliorates neovascularization, including the adenoviral-responsive gene products described herein that inhibit angiogenesis. One of ordinary skill in the art will understand that complete prevention or amelioration of neovascularization is not required in order to realize a desired biological effect. Therefore, the inventive method contemplates both partial and complete prevention and amelioration of angiogenesis. An inhibitor of angiogenesis includes, for instance, an anti-angiogenic factor, an anti-sense molecule specific for an angiogenic factor, a ribozyme, a small interfering RNA (siRNA, an RNA interfering molecule), a receptor for an angiogenic factor, and an antibody that binds a receptor for an angiogenic factor.

Anti-angiogenic factors include, for example, pigment epithelium-derived factor, angiostatin, vasculostatin, endostatin, platelet factor 4, heparinase, interferons (e.g., INFα), tissue inhibitor of metalloproteinase 3 (TIMP3), and the like. Such factors prevent the growth of new blood vessels, promote vessel maturation, inhibit permeability of blood vessels, inhibit the migration of endothelial cells, and the like. Various anti-angiogenic factors are described in International Patent Application WO 02/22176. One of ordinary skill in the art will appreciate that any anti-angiogenic factor can be modified or truncated and retain anti-angiogenic activity. As such, active fragments of anti-angiogenic factors (i.e., those fragments having biological activity sufficient to inhibit angiogenesis) are also suitable.

An anti-sense molecule specific for an angiogenic factor should generally be substantially identical to at least a portion, preferably at least about 20 continuous nucleotides, of the nucleic acid encoding the angiogenic factor to be inhibited, but need not be identical. The anti-sense nucleic acid molecule can be designed such that the inhibitory effect applies to other proteins within a family of genes exhibiting homology or substantial homology to the nucleic acid. The introduced anti-sense nucleic acid molecule also need not be full-length relative to either the primary transcription product or fully processed mRNA. Generally, higher homology can be used to compensate for the use of a shorter sequence. Furthermore, the anti-sense molecule need not have the same intron or exon pattern, and homology of non-coding segments will be equally effective. Antisense phosphorothiotac oligodeoxynucleotides (PS-ODNs) is exemplary of an anti-sense molecule specific for an angiogenic factor. Also suitable are other RNA interfering agents, such as siRNA (see, e.g., Chui et al., Mol. Cell., 10(3), 549-61 (2002)).

Ribozymes can be designed that specifically pair with virtually any target RNA and cleave the phosphodiester backbone at a specific location, thereby functionally inactivating the target RNA. In carrying out this cleavage, the ribozyme is not itself altered and is, thus, capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is described in Haseloffet al., Nature, 334, 585-591 (1988). Preferably, the ribozyme comprises at least about 20 continuous nucleotides complementary to the target sequence on each side of the active site of the ribozyme.

Receptors specific for angiogenic factors inhibit neovascularization by sequestering growth factors away from functional receptors capable of promoting a cellular response. For example, Flt and Flk receptors (e.g., soluble flt (sflt)), as well as VEGF-receptor chimeric proteins, compete with VEGF receptors on vascular endothelial cells to inhibit endothelial cell growth (Aiello, PNAS, 92, 10457 (1995)). Also contemplated are growth factor-specific antibodies and fragments thereof (e.g., Fab, F(ab′)₂, and Fv) that neutralize angiogenic factors or bind receptors for angiogenic factors.

The expression vector encoding the adenoviral-responsive gene product or another co-administered expression vector can comprise a nucleic acid sequence encoding a vessel maturation factor. Many ocular disorders involve leakage of blood products through vessels, which can cloud vision and induce an immune response within the layers of the eye. Vessel maturation factors reduce the amount of vascular leakage and, therefore, are useful in treating, for example, exudative ocular disorders. Vessel maturation factors include, but are not limited to, angiopoietins (Ang, e.g., Ang-1 and Ang-2), tumor necrosis factor-alpha (TNF-α), midkine (MK), COUP-TFII, hepatic growth factor (HGF), and heparin-binding neurotrophic factor (HBNF, also known as heparin binding growth factor).

The invention also contemplates delivery of a nucleic acid sequence encoding at least one neurotrophic agent (or neurotrophic factor) to ocular cells. Neurotrophic factors are thought to be responsible for the maturation of developing neurons and for maintaining adult neurons. Thus, the method of the invention can be used to inhibit or reverse neural cell degeneration and death not associated with neovascular diseases. Neurotrophic factors are divided into three subclasses: neuropoietic cytokines; neurotrophins; and the fibroblast growth factors. Ciliary neurotrophic factor (CNTF) is exemplary of neuropoietic cytokines. CNTF promotes the survival of ciliary ganglionic neurons and supports certain neurons that are NGF-responsive. Neurotrophins include, for example, brain-derived neurotrophic factor and nerve growth factor, perhaps the best characterized neurotrophic factor. Other neurotrophic factors suitable for being encoded by the nucleic acid sequence of the inventive method include, for example, transforming growth factors, glial cell-line derived neurotrophic factor, neurotrophin 3, neurotrophin 4/5, and interleukin 1-β. Neurotrophic factors associated with angiogenesis, such as aFGF and bFGF, are less preferred. The neurotrophic factor can also be a neuronotrophic factor, e.g., a factor that enhances neuronal survival. It has been postulated that neurotrophic factors can actually reverse degradation of neurons. Such factors, conceivably, are useful in treating the degeneration of neurons associated with vision loss. Neurotrophic factors function in both paracrine and autocrine fashions, making them ideal therapeutic agents. A nucleic acid sequence used in the context of the invention can encode both an inhibitor of angiogenesis and a neurotrophic factor. More preferably, the nucleic acid sequence encodes at least one factor comprising both anti-angiogenic and neurotrophic properties. Most preferably, the factor comprising both anti-angiogenic and neurotrophic properties is PEDF.

PEDF, also named early population doubling factor-1 (EPC-1), is a secreted protein having homology to a family of serine protease inhibitors named serpins. PEDF is made predominantly by retinal pigment epithelial cells and is detectable in most tissues and cell types of the body. PEDF has been observed to induce differentiation in retinoblastoma cells and enhance survival of neuronal populations (Chader, Cell Different., 20, 209-216 (1987)). Factors that enhance neuronal survival under adverse conditions, such as PEDF, are termed “neuronotrophic,” as described herein. PEDF further has gliastatic activity, or has the ability to inhibit glial cell growth. As discussed above, PEDF also has anti-angiogenic activity. Anti-angiogenic derivatives of PEDF include SLED proteins, discussed in WO 99/04806. It has also been postulated that PEDF is involved with cell senescence (Pignolo et al., J. Biol. Chem., 268(12), 8949-8957 (1998)). Ideally, the inventive method comprises administering PEDF in combination with the adenoviral-responsive gene product. PEDF for use in the inventive method can be derived from any source, and is further characterized in U.S. Pat. No. 5,840,686 and International Patent Applications WO 93/24529 and WO 99/04806. Desirably, the invention comprises administering to the eye an adenoviral vector comprising the nucleic acid sequence set forth in SEQ ID NO: 1.

An expression vector, e.g., an adenoviral or an adeno-associated viral vector, also can comprise a nucleic acid sequence encoding a protein fragment, such as a therapeutic fragment of an adenoviral-responsive gene product, an inhibitor of angiogenesis, or a neurotrophic factor. One of ordinary skill in the art will appreciate that any inhibitor of angiogenesis or neurotrophic factor, e.g., PEDF, can be modified or truncated and retain anti-angiogenic or neurotrophic activity. As such, coding sequences for therapeutic fragments (i.e., those fragments having biological activity sufficient to, for example, inhibit angiogenesis or promote neuron survival) also are suitable for incorporation into the expression vector. Also suitable for incorporation into the expression vector are nucleic acid sequences comprising substitutions, deletions, or additions, but which encode a functioning adenoviral-responsive gene product, inhibitor of angiogenesis, or neurotrophic factor or a therapeutic fragment of any of the foregoing. A functioning inhibitor of angiogenesis or a therapeutic fragment thereof prevents or ameliorates neovascularization. A functioning neurotrophic factor or a therapeutic fragment thereof desirably promotes neuronal cell differentiation, inhibits glial cell proliferation, and/or promotes neuronal cell survival. A functioning adenoviral-responsive gene product for use in the invention mediates a desired biological effect in the eye. The ordinarily skilled artisan has the ability to determine whether a modified therapeutic factor or a fragment thereof has a desired biological activity, such as neurotrophic and anti-angiogenic therapeutic activity using routine laboratory assays. For example, neuronal cell differentiation and survival assays (see, for example, U.S. Pat. No. 5,840,686), the mouse ear model of neovascularization, or the rat hindlimb ischemia model is useful for evaluating anti-angiogenesis and/or neuroprotective activities.

The invention also contemplates the use of nucleic acid sequences encoding chimeric or fusion peptides. Through recombinant DNA technology, scientists have been able to generate fusion proteins that contain the combined amino acid sequence of two or more proteins. The ordinarily skilled artisan can fuse the active domains of two or more factors to generate chimeric peptides with desired activity. A fusion protein, such as a fusion protein comprising an adenoviral-responsive gene product, an anti-angiogenic factor, or neurotrophic factor or a therapeutic fragment thereof and for example, a moiety that stabilizes peptide conformation, can be used in the context of the invention. The chimeric peptide can comprise the entire amino acid sequences of two or more peptides or, alternatively, can be constructed to comprise portions of two or more peptides (e.g., 10, 20, 50, 75, 100, 400, 500, or more amino acid residues).

The method of the invention can be part of a treatment regimen involving other therapeutic modalities. It is appropriate, therefore, if the ocular-related disorder, namely ocular neovascularization or age-related macular degeneration, has been treated, is being treated, or will be treated with any of a number of additional ocular therapies, such as drug therapy, panretinal therapy, thermotherapy, radiation therapy, or surgery. The surgery can comprise, for instance, macular translocation, removal of subretinal blood, or removal of subretinal choroidal neovascular membrane. The expression vector is preferably administered intraocularly for the prophylactic or therapeutic treatment of an ocular-related disorder, e.g., age-related macular degeneration or persistent or recurrent ocular neovascularization, which is also treated with drugs, surgery, laser photocoagulation, and/or photodynamic therapies.

The expression vector of the inventive method is delivered to the eye, wherein the expression vector transduces host cells. Delivery to the eye can be achieved by administering the expression vector to any component of the ocular apparatus (e.g., eye globe, layers of the eye globe, muscles or connective tissue associated with the eye, etc.), such that the gene product is produced and delivered to target ocular cells. Ocular cells include, but are not limited to, cells of neural origin, cells of all layers of the retina, especially retinal pigment epithelial cells, glial cells, pericytes, endothelial cells, iris epithelial cells, corneal cells, ciliary epithelial cells, Mueller cells, astrocytes, muscle cells surrounding and attached to the eye (e.g., cells of the lateral rectus muscle), fibroblasts (e.g., fibroblasts associated with the episclera), orbital fat cells, cells of the sclera and episclera, connective tissue cells, muscle cells, cells of the trabecular meshwork, fibroblasts, and vascular endothelial cells. In that a great deal of retinal damage occurs as a result of edema, thickening of underlying membranes, and build-up of metabolic byproducts, the expression vector can be administered to an area of vascular leakage.

The expression vector desirably is administered in a pharmaceutical composition, which comprises a pharmaceutically acceptable (e.g., physiologically acceptable) carrier and the expression vector(s). Any suitable pharmaceutically acceptable carrier can be used within the context of the invention, and such carriers are well known in the art. The choice of carrier will be determined, in part, by the particular site to which the composition is to be administered and the particular method used to administer the composition.

Suitable formulations for the pharmaceutical composition include aqueous and non-aqueous solutions, isotonic sterile solutions, which can contain anti-oxidants, buffers, bacteriostats, and solutes that render the formulation isotonic with the blood or intraocular fluid of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. Preferably, the pharmaceutically acceptable carrier is a buffered saline solution. More preferably, the expression vector is administered in a pharmaceutical composition formulated to protect and/or stabilize the expression vector from damage prior to administration. For example, the pharmaceutical composition can be formulated to reduce loss of the expression vector on devices used to prepare, store, or administer the expression vector, such as glassware, syringes, pellets, slow-release devices, pumps, or needles. The pharmaceutical composition can be formulated to decrease the light sensitivity and/or temperature sensitivity of the expression vector. To this end, the pharmaceutical composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. In one embodiment, the formulation comprises Tris base (10 mM), NaCl (75 mM), MgCl.6H₂O (1 mM), polysorbate 80 (0.0025%) and trehalose dehydrate (5%). Use of such a pharmaceutical composition will extend the shelf life of the vector, facilitate administration, and increase the efficiency of the inventive methods. In this regard, a pharmaceutical composition also can be formulated to enhance transduction efficiency. Suitable compositions are further described in U.S. Pat. Nos. 6,225,289 and 6,514,943.

In addition, the expression vector, e.g., viral vector, of the invention can be present in a composition with other therapeutic or biologically-active agents. For example, therapeutic factors useful in the treatment of a particular indication can be present. For instance, the expression vector can be administered in combination with PEDF or sflt. Indeed, any of the inhibitors of angiogenesis or neurotrophic factors described herein can be co-administered in protein form. If treating vision loss, hyaluronidase can be added to a composition to, for example, affect the break down of blood and blood proteins in the vitreous of the eye. Factors that control inflammation, such as ibuprofen or steroids, can be part of the composition to reduce swelling and inflammation associated with in vivo administration of the viral vector and ocular distress. Inflammation also can be controlled by down-regulating the effects of cytokines involved in the inflammation process (e.g., TNFα). Alternatively, agonists for chemokines which control inflammation (e.g., TGFβ) can be included to reduce the harmful effects of inflammation. Immune system suppressors can be administered in combination with the inventive method to reduce any immune response to the vector itself or associated with an ocular disorder. Anti-angiogenic factors, such as soluble growth factor receptors (sflt), growth factor antagonists (e.g., angiotensin), an anti-growth factor antibody (e.g., Lucentis™), Squalamine (an aminosterol), and the like also can be part of the composition, as well as neurotrophic factors. Similarly, vitamins and minerals, anti-oxidants, and micronutrients can be co-administered. Antibiotics, i.e., microbicides and fungicides, can be present to reduce the risk of infection associated with gene transfer procedures and other disorders. Ligands for nuclear receptors such as thyroid hormones, retinoids, specific prostaglandins, estrogen hormone, glucocorticoids, and their analogues can be part of the composition. Small molecule agonists for the PEDF receptor also can be included in the composition. Such small molecule agonists can amplify the therapeutic effect of the inventive method. Suitable drugs for inclusion in the composition include, but are not limited to, a prostaglandin analogue, a beta-blocker (as commonly used for glaucoma treatment), hyaluronidase (e.g., Vitrase™ available from Allergan), pegaptanib sodium (e.g., Macugen™), tetrahydrozoline hydrochloride (e.g., Visine™), dorzolamide hydrochloride (Cosopt™ and Truspot™), and an alpha-2-adrenergic agonist (e.g., Alphagan™). Alternatively, these compounds can be administered separately.

One skilled in the art will appreciate that suitable methods, i.e., invasive and noninvasive methods, of administering an expression vector to the eye are available. Although more than one route can be used to administer a particular expression vector to an eye, such as a human eye, a particular route can provide a more immediate and more effective reaction than another route. Accordingly, the described routes of administration are merely exemplary and are in no way limiting.

Any route of administration is appropriate so long as the expression vector transduces a host cell. The expression vector can be appropriately formulated and administered in the form of an injection, eye lotion, ointment, implant and the like. An expression vector can be applied, for example, topically, intracamerally, subconjunctivally, intraocularly, retrobulbarly, periocularly (e.g., subtenon delivery), subretinally, intravitreously, or suprachoroidally for direct administration to the eye. In certain cases, it may be appropriate to administer multiple applications and employ multiple routes, e.g., subretinal and intravitreous, to ensure sufficient exposure of ocular cells to the expression vector. Multiple applications of the expression vector may also be required to achieve the desired effect.

Depending on the particular case, it may be desirable to non-invasively administer the expression vector to a patient. For instance, if multiple surgeries have been performed, the patient displays low tolerance to anesthetic, or if other ocular-related disorders exist, topical administration of the expression vector may be most appropriate. Topical formulations are well known to those of skill in the art. Such formulations are suitable in the context of the invention for application to the skin. The use of patches, corneal shields (see, e.g., U.S. Pat. No. 5,185,152), and ophthalmic solutions (see, e.g., U.S. Pat. No. 5,710,182) and ointments, e.g., eye drops is also within the skill in the art. The expression vector also can be administered non-invasively using a needleless injection device, such as the Biojector 2000 Needle-Free Injection Management System® available from Bioject, Inc.

The expression vector can be present in or on a device that allows controlled or sustained release of the expression vector, such as an ocular sponge, meshwork, mechanical reservoir, or mechanical implant. Implants (see, e.g., U.S. Pat. Nos. 4,853,224, 4,997,652, and 5,443,505), and devices (see, e.g., U.S. Pat. Nos. 4,863,457, 5,098,443, 5,554,187, and 5,725,493), such as an implantable device, e.g., a mechanical reservoir, an intraocular device or an extraocular device with an intraocular conduit, or an implant or a device comprised of a polymeric composition, are particularly useful for ocular administration of the expression vector. An expression vector also can be administered in the form of sustained-release formulations (see, e.g., U.S. Pat. No. 5,378,475) comprising, for example, gelatin, chondroitin sulfate, a polyphosphoester, such as bis-2-hydroxyethyl-terephthalate (BHET), or a polylactic-glycolic acid.

Alternatively, the expression vector can be administered using invasive procedures, such as, for instance, intravitreal injection or subretinal injection, optionally preceded by a vitrectomy, or periocular (e.g., subtenon) delivery. The expression vector can be injected into different compartments of the eye, e.g., the vitreal cavity or anterior chamber. Pharmaceutically acceptable carriers for injectable compositions are well-known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4^{th} ed., pages 622-630 (1986)). Although less preferred, the expression vector can also be administered in vivo by particle bombardment, i.e., a gene gun.

Preferably, the expression vector is administered via an ophthalmologic instrument for delivery to a specific region of an eye. Use of a specialized ophthalmologic instrument ensures precise administration of the expression vector while minimizing damage to adjacent ocular tissue. A preferred ophthalmologic instrument is a combination of forceps and subretinal needle or sharp bent cannula.

While intraocular injection is preferred, injectable compositions also can be administered intramuscularly, intravenously, intraarterially, intraperitoneally, parenterally, systemically, or subcutaneously. Expression vectors also can be administered intratracheally, orally, trans-dermally, or intranasally. Preferably, any expression vector administered to a patient using these routes of administration in the context of the invention is specifically targeted to ocular cells. As discussed herein, an expression vector can be modified to alter the binding specificity or recognition of an expression vector for a receptor on a potential host cell. With respect to adenovirus, such manipulations can include deletion of regions of the fiber, penton, or hexon, insertions of various native or non-native ligands into portions of the coat protein, and the like. One of ordinary skill in the art will appreciate that parenteral administration can require large doses or multiple administrations to effectively deliver the expression vector to the appropriate host cells.

One of ordinary skill in the art will also appreciate that dosage and routes of administration can be selected to minimize loss of expression vector due to a host's immune system. For example, for transducing ocular cells in vivo, it can be advantageous to administer to a host a null expression vector (i.e., an expression vector not comprising the nucleic acid sequence encoding the adenoviral-responsive gene product) prior to performing the inventive method. Prior administration of null expression vectors can serve to create an immunity (e.g., tolerance) in the host to the expression vector, thereby decreasing the amount of vector cleared by the immune system.

The dose of expression vector administered to an animal, particularly a human, in accordance with the invention should be sufficient to affect the desired response in the animal over a reasonable time frame. One skilled in the art will recognize that dosage will depend upon a variety of factors, including the age, species, the pathology in question, and condition or disease state. Dosage also depends on the gene product, e.g., inhibitor of angiogenesis and/or neurotrophic factor, to be expressed, as well as the amount of ocular tissue to be transduced and/or about to be affected or actually affected by the ocular-related disease. The size of the dose also will be determined by the route, timing, and frequency of administration as well as the existence, nature, and extent of any adverse side effects that might accompany the administration of a particular expression vector and the desired physiological effect. It will be appreciated by one of ordinary skill in the art that various conditions or disease states, in particular, chronic conditions or disease states, may require prolonged treatment involving multiple administrations.

Suitable doses and dosage regimens can be determined by conventional range-finding techniques known to those of ordinary skill in the art. Preferably, about 10⁶ viral particles to about 10¹² viral particles are delivered to the patient. In other words, a pharmaceutical composition can be administered that comprises an expression vector concentration of from about 10⁶ particles/ml to about 10¹² particles/ml (including all integers within the range of about 10⁶ particles/ml to about 10¹² particles/ml), preferably from about 10¹⁰ particles/ml to about 10¹² particles/ml, and will typically involve the intraocular administration of from about 0.1 μl to about 100 μl of such a pharmaceutical composition per eye. In some instances, an injection can comprise from about 0.5 mL to about 1 mL of pharmaceutical composition. Ideally, a dose of about 1×10⁶, about 1×10^{6.5}, about 1×10⁷, about 1×10^{7.5}, about 1×10⁸, about 1×10^{8.5}, about 1×10⁹, or about 1×10^{9.5} particles of adenoviral vector (e.g., about 3×10⁷, 3×10⁸, or 3×10⁹ particles of adenoviral vector), or a dose within a range between two of the aforementioned numbers, is administered per eye to a patient via intravitreal injection. Alternatively, the adenoviral vector of the inventive method is administered subretinally in a dose of about 1×10⁵, about 1×10^{5.5}, about 1×10⁶, about 1×10^{6.5}, about 1×10⁷, about 1×10^{7.5}, about 1×10⁸, or about 1×10^{8.5} particles per eye, or in a dose within a range between two of the aforementioned numbers. When administered periocularly, the dose of adenoviral vector administered preferably is about 1×10⁷, about 1×10^{7.5}, about 1×10⁸, about 1×10^{8.5}, about 1×10⁹, about 1×10^{9.5}, about 1×10¹⁰, about 1×10^{10.5}, about 1×10¹¹, about 1×10^{11.5}, or about 1×10¹² particles per eye, or in a dose within a range between two of the aforementioned numbers. When the expression vector is a plasmid, preferably about 0.5 ng to about 1000 μg of DNA is administered. More preferably, about 0.1 μg to about 500 μg is administered; even more preferably about 1 μg to about 100 μg of DNA is administered. Most preferably, about 50 μg of DNA is administered per eye. Of course, other routes of administration may require smaller or larger doses to achieve a therapeutic effect. Any necessary variations in dosages and routes of administration can be determined by the ordinarily skilled artisan using routine techniques known in the art.

If inducing a stress response in the eye, the expression vector can be administered any time after induction. It is desirable to administer the expression vector after inducing the stress response such that transduction of host cells is enhanced and expression of the nucleic acid sequence (i.e., transcription) is increased as compared to an expression vector administered in the absence of inducing a stress response in the eye. Preferably, the expression vector is administered within 3 months (e.g., within 2 months) of inducing a stress response in the eye (e.g., exposing the eye to photocoagulation or photodynamic therapy). More preferably, the expression vector is administered within 28 days (e.g., within 21 days or within 14 days) of inducing a stress response in the eye. Also preferably, the expression vector is administered to the eye within 7 days of (e.g., 1, 2, 3, 4, 5, 6, or 7 days after) inducing a stress response in the eye. Also preferably, the expression vector is administered within 3 days (e.g., 1, 2, or 3 days) or within 1 day of inducing the stress response in the eye.

In some embodiments, it is advantageous to administer two or more (i.e., multiple) doses of the expression vector comprising a nucleic acid sequence encoding a gene product to achieve the desired biological response. The inventive method provides for multiple applications of the expression vector. For example, at least two applications of an expression vector can be administered to the same eye. Preferably, the multiple doses are administered while retaining gene expression above background levels. Also preferably, two applications or more of the expression vector is administered within about 30 days or more. More preferably, two or more applications are administered to the same eye within about 90 days or more. However, three, four, five, six, or more doses can be administered in any time frame (e.g., 2, 7, 10, 14, 21, 28, 35, 42, 49, 56, 63, 70, 77, 85 or more days between doses) so long as gene expression occurs (and preferably an ocular disorder is inhibited or ameliorated).

As discussed herein, the expression vector of the inventive method comprises a nucleic acid sequence that encodes an adenoviral-responsive gene product. The nucleic acid sequence can encode multiple, i.e., two, three, or more, adenoviral-responsive gene products, or comprise additional transgenes. In a preferred embodiment, the expression vector additionally contains a nucleic acid sequence encoding PEDF, ciliary neurotrophic factor (CNTF), and/or sflt. Multiple gene products can be operably linked to different promoters. Multiple gene products can be encoded by multiple expression vectors, which are administered to the eye and produced within a target cell.

A transgene encoding a marker protein, such as green fluorescent protein or luciferase, can be incorporated into the expression vector. Such marker proteins are useful in vector construction and determining vector migration. Marker proteins also can be used to determine points of injection or treated ocular tissues in order to efficiently space injections of the expression vector to provide a widespread area of treatment, if desired.

The inventive method also can involve the co-administration of other pharmaceutically active compounds. By “co-administration” is meant administration before, concurrently with, e.g., in combination with the expression vector in the same formulation or in separate formulations, or after administration of the expression vector as described above. Any of the exogenous materials, drugs, proteins, and the like described herein can be co-administered with the expression vector as adjuvant therapy. For example, factors that control inflammation, such as ibuprofen or steroids, can be co-administered to reduce swelling and inflammation associated with intraocular administration of the expression vector. Immunosuppressive agents can be co-administered to reduce inappropriate immune responses related to an ocular disorder or the practice of the inventive method. Anti-angiogenic factors, such as soluble growth factor receptors, growth factor antagonists, i.e., angiotensin, and the like, can also be co-administered, as well as neurotrophic factors. In addition, the expression vector of the inventive method can be administered with anti-proliferative agents such as siRNA, aptamers, or antibodies which sequester or inactivate angiogenic factors such as, for example, VEGF. Similarly, vitamins and minerals, anti-oxidants, and micronutrients can be co-administered. Antibiotics, i.e., microbicides and fungicides, can be co-administered to reduce the risk of infection associated with ocular procedures and some ocular-related disorders. Other therapeutics for ocular disorders can be administered in conjunction with the inventive method. For example, Visudyne® (Novartis), Macugen™ (Pfizer), Retaane™ (Alcon), Lucentis™ (Genentech/Novartis), Squalamine (Genaera), Cosopt, and Alphagan can be formulated with the expression vector or can be administered separately before, during, or after administration of the expression vector to the animal.

The following examples further illustrate the invention but, of course, should not be construed as in any way limiting its scope.

### EXAMPLE 1

It has been demonstrated that empty adenoviral vectors have anti-neovascular activity in addition to that of the anti-angiogenic factor, pigment epithelium-derived factor (Mori et al., J. Cell Physiol., 188(2), 253-63 (August 2001)). This example demonstrates a method of inhibiting angiogenesis by administering an adenoviral vector containing no therapeutic transgene (AdNull vector), as well as a method of identifying adenoviral-responsive gene products associated with angiogenesis inhibition.

C57BL/6 mouse pups were exposed to 75% oxygen from postnatal day (P) 7 to 12 and then returned to room air. Intravitreous (IV) injection of 1×10⁹ particles of serotype 5, E1−, E3−, E4+adenoviral vector, serotype 5, E1−, E3−, E4− adenoviral vector, or vehicle was performed on P10. Histopathological evaluation, quantitative analysis of retinal neovascularization, microarray, and qRT-PCR analysis was performed on eyes from each group.

Retinal neovascularization was significantly reduced after IV injection of the E1−, E3−, E4+ adenoviral vectors and E1-, E3−, E4− adenoviral vectors (p<0.001, 83% reduction and p<0.01, 43% reduction, respectively). Using stringent statistical analyses, 48 genes were determined to be preferentially expressed in vector-injected eyes including, but not limited to, CC/CXC chemokines and ESTs.

The results of this example demonstrate that retinal angiogenesis can be inhibited by intravitreously administering an AdNull vector. Microarray and qRT-PCR analysis identified 48 over-expressed genes that may contribute to the anti-angiogenic activity of the AdNull vector. These data confirm that adenoviral-responsive gene products provide an anti-angiogenic effect, thereby effecting treatment of ocular-related disorders.

### EXAMPLE 2

This example demonstrates the neuroprotective effect associated with the administration of E1−, E3−, E4− adenoviral vectors not encoding a transgene in a murine model of light-induced photoreceptor degeneration. This example further demonstrates a method of identifying adenoviral-responsive gene products associated with inhibition of photoreceptor cell loss.

Adenoviral vector delivery of pigment epithelium-derived factor (AdPEDF) rescued photoreceptors from light-induced cell death (Imai et al., J. Cell Physiol., 202(2), 570-78 (2005)). Empty adenoviral vectors, i.e., adenoviral vectors not comprising a therapeutic transgene (AdNull vectors), also displayed a neuroprotective effect, although the effect was significantly less than AdPEDF. Dark-adapted BALB/c mice, aged 6-8 weeks, were exposed to standardized, intense fluorescent light for 12, 96, or 144 hours. Prior to dark-adaptation, all mice received intravitreous injection (IV) of 1×10⁹ particles of an empty E1−, E3−, E4−adenoviral vector in one eye. An IV injection of vehicle was administered to the other eye. Following light challenge of 96 and 144 hours, histopathological analysis, including quantitative photoreceptor cell counts (number of photoreceptors per 50 μm), was conducted. Semi-quantitative assessment of mRNA for the following apoptosis-related genes was performed on eyes following 12 hours light exposure using qRT-PCR: p50, p65, IkBa, caspase-1, caspase-3, Bad, Jun, Bax, Bak, Bcl-2, fos, and p53.

After 96 hours of light exposure, photoreceptor cell density for E1−, E3−, E4− adenoviral vector-injected eyes and vehicle-injected eyes were 87.5±9.5 and 79.3±9.0, respectively (p=0.14) . After 144 hours of light exposure, photoreceptor cell density was preserved in vector-injected eyes as compared to vehicle-injected eyes, with values of 68.9 ±9.9 and 49.2±4.1, respectively, (p=0.016). Relative mRNA levels of p50, caspase-1, and Bcl-2 differed significantly between vector- and vehicle-injected eyes (p=0.026, p=0.034, p=0.028, respectively). The expression levels of other evaluated apoptosis-related genes were not significantly different from baseline.

The results of this example demonstrate that administration of an adenoviral vector, which upregulates production of adenoviral-responsive gene products, increases photoreceptor cell survival caused by intense light exposure. Changes in gene expression suggest that the protective effect involves the NFkB/caspase-1 pathway, as well as an anti-apoptotic effect of Bcl-2. The upregulated gene products can be used to reduce photoreceptor loss in response to external stimuli, and are candidates for co-therapies in conjunction with the anti-angiogenic agent PEDF.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of prophylactically or therapeutically treating an animal for an ocular-related disorder, wherein the method comprises administering to an eye of the animal an expression vector comprising a nucleic acid sequence encoding an adenoviral-responsive gene product such that the expression vector transduces a host cell and the nucleic acid sequence is expressed to produce the adenoviral-responsive gene product to treat prophylactically or therapeutically the ocular-related disorder.

2. The method of claim 1, wherein the ocular-related disorder is selected from the group consisting of ocular neovascularization, age-related macular degeneration, retinal tumors, diabetic retinopathy, macular edema, glaucoma, and retinal degenerative disease.

3. A method of reducing or inhibiting angiogenesis in an eye, wherein the method comprises administering to the eye an expression vector comprising a nucleic acid sequence encoding an adenoviral-responsive gene product such that the expression vector transduces a host cell and the nucleic acid sequence is expressed to produce the adenoviral-responsive gene product thereby reducing or inhibiting angiogenesis in the eye.

4. A method of reducing or inhibiting photoreceptor cell loss in an eye, wherein the method comprises administering to the eye an expression vector comprising a nucleic acid sequence encoding an adenoviral-responsive gene product such that the expression vector transduces a host cell and the nucleic acid sequence is expressed to produce the adenoviral-responsive gene product thereby reducing or inhibiting photoreceptor cell loss in the eye.

5. The method of claim 1, wherein the expression vector is a viral vector.

6. The method of claim 1, wherein the expression vector is an adenoviral vector.

7. The method of claim 6, wherein the adenoviral vector is a human adenoviral vector.

8. The method of claim 6, wherein the adenoviral vector is replication-deficient.

9. The method of claim 8, wherein the adenoviral vector is deficient in at least one replication-essential gene function of the E1 region of the adenoviral genome of the adenoviral vector.

10. The method of claim 9, wherein the adenoviral vector is deficient in at least one replication-essential gene function of the E4 region of the adenoviral genome of the adenoviral vector.

11. The method of claim 1, wherein the adenoviral-responsive gene product is a CC chemokine, a CXC chemokine, p50, caspase-1, or Bcl-2.

12. The method of claim 1, wherein the method further comprises administering to the eye a nucleic acid encoding an inhibitor of angiogenesis or a neuroprotective factor.

13. The method of claim 12, wherein the inhibitor of angiogenesis is soluble flt or pigment epithelium-derived factor (PEDF).

14. The method of claim 12, wherein the nucleic acid encoding an inhibitor of angiogenesis or a neuroprotective factor and the nucleic acid sequence encoding the adenoviral-responsive gene product are present in the same expression vector.

15. The method of claim 1, wherein the expression vector is administered topically, subconjunctivally, retrobulbarly, periocularly, intravitreously, subretinally, suprachoroidally, or intraocularly.

16. The method of claim 15, wherein the expression vector is administered intravitreously.


SEQUENCE LISTING (1 sequence)

SEQ ID NO: 1 - DNA, 33014 bp, Artificial
  Synthetic construct
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggcccgggatcggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatctttctagctagtctagactagctagactcgagagcggccgcaatcgataagcttaggggcccctggggtccagaatcttgccaatgaagagaagggcccctgtgtctgtgtccctcagtacgaagatgaaaggctggttaaggtgatagtccagcgggaaggtgaggtgggcaggctgcagccctgggctgggggtggttcccgccccatcctcgttccactcaaagccagcccggtgttccacctgagtcagcttgatgggtttgcctgtgatcttgctaaagtctggtgaatcaaacaaggattgcagcttcatctcctgcagggacttggtgacttcgccttcgtaactcagcttcagcttggggacagtgaggaccgcctgcacggtcttcagttctcggtctatgtcatgaatgaactcggaggtgaggctctcctctatcaaggtcaaattctgggtcactttcaggggcaggaagaagatgatactcatgcttccggtcaagggcagctgggcaatcttgcagctgagatctgaatccaagccatagcgtaaaacagccttagggtccgacatcatggggaccctcacggtcctctcttcatccaagtagaaatcctcgagggaagtctttctggagtcaaactttgttacccactgccccttgaagtgcgccacaccgagaaggagaatgctgatctcatcgggaatttcctttgtggacctggcgagcttccctttcatctgcgcctgcacccagttgttgatctcttgcaggtccaagcgagggttgcccgtcaggactctgggcctggtcccatatgacttttccagaggtgccacaaagctggattttatgcgcagcttcttctcaaagacgatccgggaggcactcttgaggttcttctggggggcagtgaccgtgtcaaggagctccttataggtaccatggatgtctgggctgctgatcaagtcatagtagagagcccggtgaatgatggattctgttcgctgctccgctcccagcgagagggccgagagggccgtggccacactgagaggagacaggagcacgttggtcgtggggctcatgctggatcgcacccggtacaggtcatagccgaagttggagacagccgctgccagcttgttcacggggactttgaagaaaggatcctcctcctccaccagcgcccctgtgctgtcggggtctggggagccctcctccggggggctggcagggttctggcagctgctgtgcccgaggagggctccaatgcagaggagtagcaccagggcctgcatggtggaagcttgatatcgaattctgcagtgatcagggatcccagatccgtatagtgagtcgtattaggtaccggctgcagttggacctgggagtggacacctgtggagagaaaggcaaagtggatgtcattgtcactcaagtgtatggccagatctcaagcctgccacacctcaagtgaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcccgggatcggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatcgcgatccggcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacggaccatgcgttcgacctttctcttcttttttgggcccatgatggcagatccgtatagtgagtcgtattagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagatgcatgctcgagcggccgccagtgtgatggatatctgcagaattccagcacactggcggccgttactagtggatccgagctcggtacccggccgttataacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg